(19) United States
(12) Patent Application Publication
(10) Pub. No.: US 2025/0129152 A1
(43) Pub. Date: Apr. 24, 2025

(54) MODULATORS OF INTEROCEPTION

(71) Applicant: Alberto CHIARUGI, Firenze (IT)

(72) Inventor: Alberto CHIARUGI, Firenze (IT)

(21) Appl. No.: 18/837,054

(22) PCT Filed: Jan. 19, 2023
(86) PCT No.: PCT/IT2023/050016

§ 371 Date: Aug. 8, 2024

(30) Foreign Application Priority Data
Jan. 20, 2022 (IT) 102022000000878

(51) Int. Cl. C07K 16/26; C07K 16/28

(52) U.S. Cl. CPC C07K 16/26; C07K 16/2869; C07K 2317/76

(57) ABSTRACT

Interoception is the mechanism by which an individual is informed about the status of its own body. When interoception becomes dysfunctional, it turns into a deleterious event for the health status of an individual. The use of anti-calcitonin gene-related peptide (CGRP) monoclonal antibodies for the treatment and prevention of interoception disorders is described.

### TECHNICAL FIELD

The present invention refers to the field of drugs for the treatment of disorders of interoception

### BACKGROUND ART

Interoception is the mechanism by which an individual is informed about the status of its own body. Interoception allows each individual to gather constant information of the functioning of internal organs so that, in case of dysfunction, specific responses can be activated to restore homeostasis. By so doing, interoception allows an individual to discriminate between the correct or deranged functioning of organs and functions (the health status) such as the gastrointestinal tract, the respiratory apparatus, appetite and satiety, the emotional status, sentiments, motivation as well as the presence of pain. In this perspective, perception of malaise results from interoceptive information signaling a negative health status. Interoception is therefore the opposite of exteroception that regulates the consciousness of the body in the environment.

From a neurophysiological point of view, the health status defined by interoceptive signaling is mediated by neuronal afferents belonging to both the sensory (mechanical, thermal, nociceptive) and autonomous nervous system. These peripheral afferents first project to brainstem nuclei (mostly the parabrachial nucleus) and are then redirected to the hypothalamus, amygdala, thalamus from which interoceptive information reaches the parietal, frontal and insular cortex where the consciousness and affective components of the health status are decoded.

It is well acknowledged that one of the most common homeostatic responses activated in an individual receiving a negative interoceptive information (signals of malaise) is appetite loss also known as anorexia. Even though such a response may appear paradoxical, it is, indeed, a key ancestral response in animals aimed at protecting the subject by preventing assumption of food that potentially may have caused the sickness condition. Of course, when the anorectic response is prolonged, it turns into a deleterious event for the health status, leading to excessive reduction of caloric intake, weight loss, weakness and a general derangement of the defenses of the organism. It is known that numerous hepatic, renal, gastrointestinal, immune, infective and painful disorders are invariantly associated with reduced appetite and anorexia. Likewise, a similar anorectic response is triggered by intoxication or pharmacological treatments. Among the latter, anticancer chemotherapy is very frequently associated with anorexia. Unfortunately, this type of anorexia frequently reaches such a severe intensity to critically reduce the patients' caloric intake. This, in turn, prompts therapy interruption that, inevitably, leads to chemotherapy failure and unrestricted tumor growth. It is also worth noting that the neoplastic patient is exposed to another risk of weight loss/anorexia, i.e. tumor cachexia, a serious clinical condition in which appetite loss is accompanied by loss of adipose and muscle tissues triggered by tumor-derived signals.

In light of the high frequency of food disorders related to negative interoceptive signaling and the ensuing severe clinical conditions, great effort has been directed at the identification of drugs able to restore nutritional homeostasis in these individuals.

The problem to be solved is therefore that of identifying drugs able to counteract interoceptive signaling during different types of disorders.

Even though neurochemistry of interoception is still in large part undeciphered, specific neuropeptides that modulate central processing of sensory afferents and signal malaise have been identified at the preclinical level. Neuropeptides are small (10-40 amino acids) proteinaceous molecules that play a myriad of neuronal and neuroendocrine functions within the central and peripheral nervous system. Of note, neuropeptides are released in a vesicle-dependent manner, but, at variance with classic small-molecule neurotransmitters such as glutamate, serotonin or noradrenaline, neuropeptides prompt long-lasting postsynaptic signaling that, typically, reaches neurons very distant from the site of release, thereby sustaining the so-called “volume transmission”.

Among the numerous neuropeptides, the calcitonin gene-related peptide (CGRP) is a 37 amino acid molecule with central and peripheral actions. Although the peripheral vasodilating effects of CGRP have been deeply investigated and characterized, the central effects of the neuropeptide in large part still wait to be identified. It is well known that such a lack of knowledge must be mainly ascribed to the inability of peripherally acting CGRP receptor agonists and antagonists to cross the blood-brain barrier. Such as impermeability inevitably hamper the possibility of using these tools to modulate CGRP neurotransmission and understand its role in neurophysiopathology (Nat Rev Neurol. 2018; 14:338-35). To circumvent such a technical problem, scientists have modulated CGRP neurotransmission by means of intracerebral injection of drugs regulating the neuropeptide receptor or viruses expressing proteins regulating CGRP neurotransmission. Remarkably, this technique is not clinically suitable and exploitable.

Thanks to these invasive experimental procedures, a key role of CGRP in regulating emotional functions such as responses to stress and aversive stimuli has recently emerged. As far as the aversive stimuli are concerned, it is now known that CGRP is a pivotal player of interoceptive information that reaches the parabrachial nucleus and is then redirected to the amygdala. This signaling pathway seems to play an important role is integrating sensory and autonomic afferents carrying interoceptive information in order to prompt specific homeostatic responses to different types of stresses, pain and malaise in general (Trends Neurosci. 2018; 41:280-293). On this basis, it would be theoretically possible to modulate interoception by regulating CGRP neurotransmission within the central nervous system. Unfortunately, no drugs have been so far identified that, administered in the periphery adopting a clinically suitable and exploitable method, can reach the brain and regulate CGRP-dependent interoceptive signaling within the central nervous system.

Monoclonal antibodies (mAbs) capable of inhibiting CGRP functions have been recently identified. The mAbs fremanezumab, galcanezumab and eptinezumab bind and scavenge the neuropeptide whereas erenumab binds and inhibits the neuropeptide receptor (these antibodies are collectively defined as “anti-CGRP mAbs”). These mAbs are efficacious in migraine prophylaxis. In light of the impermeability of the blood brain barrier to proteins in general and, specifically, to immunoglobulins (proteins with quaternary structure and molecular weight in the order of 150 kDa), the site of action of anti-CGRP mAbs in migraine prevention has been localized in the periphery, in particular at the level of the trigeminovascular afferents within the meninges (Nat. Rev. Neurol. 2018; 14:338-350; CNS Neurol. Disord. Drug. Targets. 2020;19:344-359). The state of the art, indeed, teaches that the anti-CGRP mAbs are unable to cross the blood brain barrier and penetrate the brain parenchyma, thereby exerting their pharmacodynamic effects exclusively in the periphery (J Neurosci. 2019; 39:6001-6011; Cephalalgia 2020; 40:229-240; Cephalalgia. 2020; 40:924-934).

Hence, the expert of the field does not find any clue in the state of the art that the anti-CGRP mAbs act within the central nervous system. Likewise, the expert of the field does not find any clue in the state of the art that the anti-CGRP mAbs regulate interoception and the homeostatic responses prompted by cerebral processing of interoceptive information.

The technical problem to be solved, therefore, is that of identifying drugs able to inhibit the actions of CGRP within the brain to modulate CGRP-dependent interoceptive information and the responses prompted by interoceptive signaling that cause malaise and negatively impact the health status of an individual.

### DISCLOSURE OF THE INVENTION

Unexpectedly, we have now found that the subcutaneous (i.e. peripheral) administration of anti-CGRP mAbs such as fremanezumab, galcanezumab, eptinezumab and erenumab is able to modulate cerebral processing of interoceptive information (i.e. malaise) in numerous experimental models of human disorders.

Specifically, we have unexpectedly found that subcutaneous injection of fremanezumab, galcanezumab, eptinezumab and erenumab reduces food aversion/anorexia, weight loss and accelerates weight body recovery in rats exposed to the chemotherapeutic drug cisplatin (6 mg/kg, intraperitoneal), as an experimental model of malaise due to antineoplastic chemotherapy (FIG. 1).

Further, we have unexpectedly found that subcutaneous injection of fremanezumab, galcanezumab, eptinezumab and erenumab reduces food aversion/anorexia and weight loss and increases spontaneous motility in mice with chronic arthritis due to injection of Freund's adjuvant as an experimental model of malaise due to chronic pain (FIG. 2).

In keeping with these findings, we have unexpectedly found that subcutaneous injection of fremanezumab, galcanezumab, eptinezumab and erenumab completely prevents weight loss and in part even the anorexia in rats subjected to laparotomy and gut manipulation as an experimental model of malaise due to abdominal surgery (FIG. 3).

Further, we have tested the anti-CGRP mAbs in a model of hepatopathy, a condition that typically prompts asthenia, weight loss and anhedonia. We have unexpectedly found that subcutaneous injection of fremanezumab, galcanezumab, eptinezumab and erenumab reduces loss of spontaneous motility as well as weight loss and triggers an increase in sweet water consumption (the latter as an index of motivational behavior and reduced anhedonia) in rats treated with carbon tetrachloride as a model of malaise due to hepatopathy (FIG. 4).

Further, we have unexpectedly found that subcutaneous injection of fremanezumab, galcanezumab, eptinezumab and erenumab counteracts weight loss and reduction of spontaneous motility in rats injected with cerulein as an experimental model of pancreatitis (FIG. 5).

Further, we have tested the anti-CGRP mAbs in rats treated with a diet containing 0.75% adenine as an experimental model of renal insufficiency. We have unexpectedly found that that subcutaneous injection of fremanezumab, galcanezumab, eptinezumab and erenumab prevents in part weight loss and anorexia in the animals (FIG. 6).

Further, we have unexpectedly found that subcutaneous injection of fremanezumab, galcanezumab, eptinezumab and erenumab in mice sensitized and re-challenged with ovalbumin as a model of asthma or chronic obstructive pulmonary disease reduces parameters of malaise such as reduction of spontaneous motility, weight loss and sweet water aversion (FIG. 7).

As additional models of malaise due to interoceptive information, we have adopted those of tumor cachexia and sepsis. FIG. 8 shows the unexpected ability the subcutaneous injection of fremanezumab, galcanezumab, eptinezumab and erenumab in reducing weight loss and counteracting both loss of muscle mass and anorexia in rats injected with Yoshida hepatoma, as a classic model of malaise due to tumor cachexia.

Similarly, we have unexpectedly found that subcutaneous injection of fremanezumab, galcanezumab, eptinezumab and erenumab reduces weight loss and anorexia in rats injected intraperitoneally with lipopolysaccharides from Salmonella typhimurium as a classic model of malaise due to sepsis/systemic immune activation (FIG. 9).

According to the invention, the anti-CGRP antibodies can be formulated and administered via the intravenous, intraarterial, intramuscular, endonasal and subcutaneous routes for the treatment of interoception disorders. The amounts of antibodies to be administered are those commonly adopted for this type of drugs, for instance 10-3000 mg adopting weekly or monthly administration.

### BRIEF DESCRIPTION OF DRAWINGS

FIG. 1. Effect of fremanezumab, galcanezumab, eptinezumab and erenumab (30 mg/kg subcutaneously, one week before the cisplatin) on food aversion/anorexia, weight loss and weight body recovery in rats exposed to cisplatin (6 mg/kg, intraperitoneal), as an experimental model of malaise due to antineoplastic chemotherapy. *p<0.05, **p<0.01, ***p<0.001, vs antibodies, ANOVA and Tukeys post hoc test.

FIG. 2. Effects of fremanezumab, galcanezumab, eptinezumab and erenumab (30 mg/kg subcutaneously, 7 days before arthritis induction) on food aversion/anorexia, weight loss and spontaneous motility in mice with chronic arthritis due to injection of Freund's adjuvant as an experimental model of malaise due to chronic pain. *p<0.05, **p<0.01, ***p<0.001 vs Arthritis, ANOVA and Tukeys post hoc test.

FIG. 3. Effects of fremanezumab, galcanezumab, eptinezumab and erenumab (30 mg/kg, subcutaneously 15 days before surgery) on weight loss and anorexia in rats subjected to laparotomy and gut manipulation as an experimental model of malaise due to abdominal surgery. *p<0.05, **p<0.01, ***p<0.001 vs Laparotomy, ANOVA and Tukeys post hoc test.

FIG. 4. Effects of fremanezumab, galcanezumab, eptinezumab and erenumab (30 mg/kg subcutaneously, 15 days before carbon tetrachloride) on spontaneous motility and weight loss as well as on sweet water consumption in rats treated with carbon tetrachloride as a model of malaise due to hepatitis/hepatopathy. *p<0.05, **p<0.01, ***p<0.001 vs Hepatitis, ANOVA and Tukeys post hoc test.

FIG. 5. Effects of fremanezumab, galcanezumab, eptinezumab and erenumab (30 mg/kg subcutaneously, 7 days before cerulein treatment) on weight loss and spontaneous motility in rats injected with cerulein as an experimental model of pancreatitis. ***p<0.001 vs Pancreatitis, ANOVA and Tukeys post hoc test.

FIG. 6. Effects of fremanezumab, galcanezumab, eptinezumab and erenumab (30 mg/kg subcutaneously, 12 days before exposure to the nephrotoxic diet) on weight loss and anorexia in rats exposed to a diet containing 0.75% adenine as an experimental model of renal insufficiency. *p<0.05, **p<0.01, ***p<0.001 vs Kidney Failure, ANOVA and Tukeys post hoc test.

FIG. 7. Effects of fremanezumab, galcanezumab, eptinezumab and erenumab (30 mg/kg subcutaneously, 12 days before antigen rechallenge) on spontaneous motility, weight loss and sweet water consumption of mice sensitized and re-challenged with ovalbumin as a model of asthma or chronic obstructive pulmonary disease. *p<0.05, **p<0.01, ***p<0.001 vs Kidney Failure, ANOVA and Tukeys post hoc test.

FIG. 8. Effects of fremanezumab, galcanezumab, eptinezumab and erenumab (30 mg/kg subcutaneously, 15 days before tumor injection) on weight loss, muscle mass and anorexia in rats injected with Yoshida hepatoma, as classic model of malaise due to tumor cachexia. *p<0.05, **p<0.01, ***p<0.001. ****p<0.0001 vs Hepatoma, ANOVA and Tukeys post hoc test.

FIG. 9. Effects of fremanezumab, galcanezumab, eptinezumab and erenumab (30 mg/kg subcutaneously, 15 days before lipopolysaccharides injection) on weight loss and anorexia in rats injected intraperitoneally with lipopolysaccharides from Salmonella typhimurium as a classic model of malaise due to sepsis/systemic immune activation. *p<0.05, **p<0.01, ***p<0.001 vs Sepsis, ANOVA and Tukeys post hoc test.

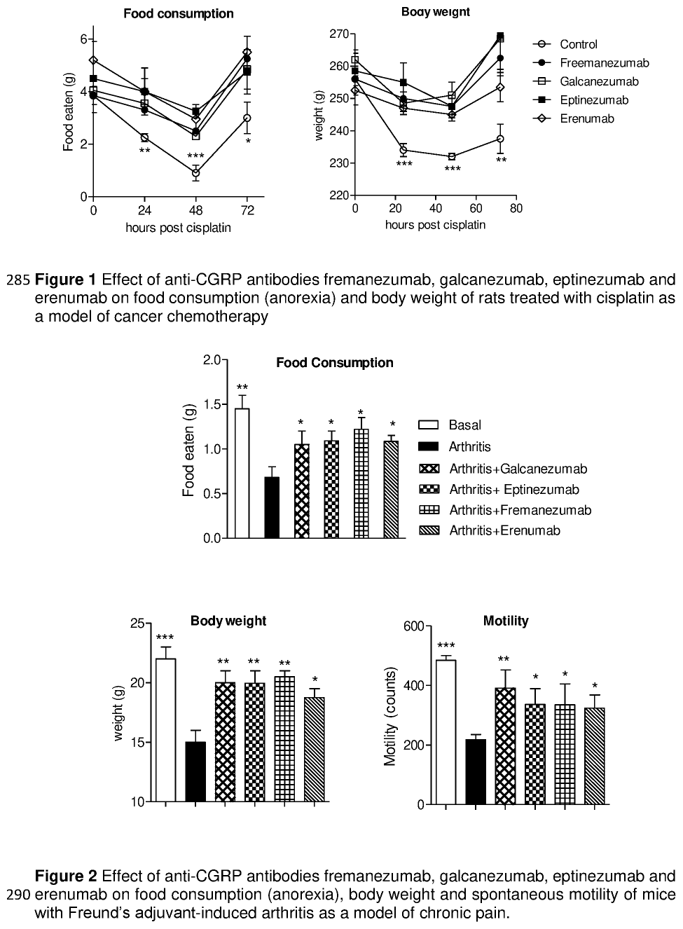

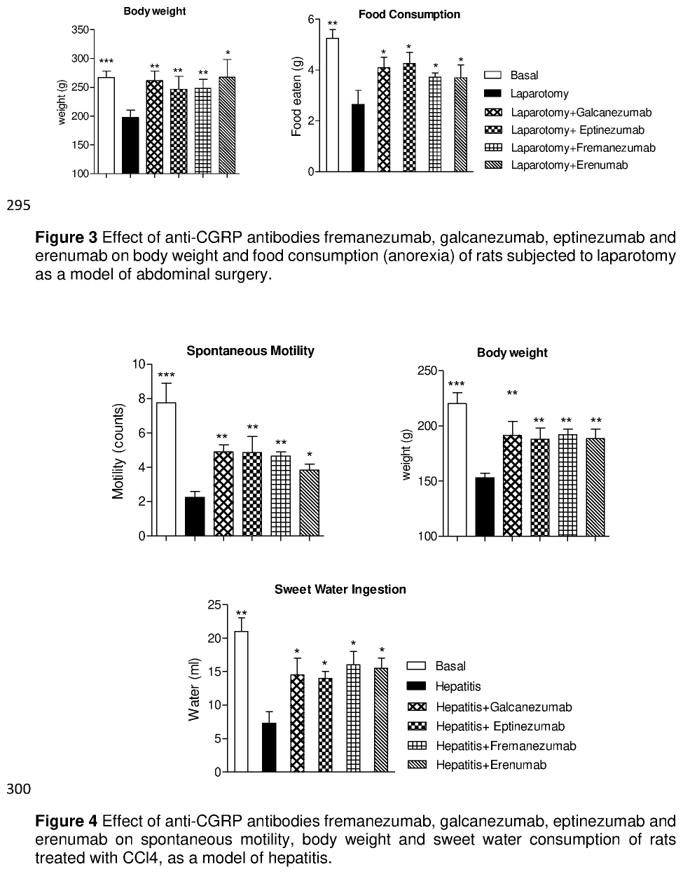

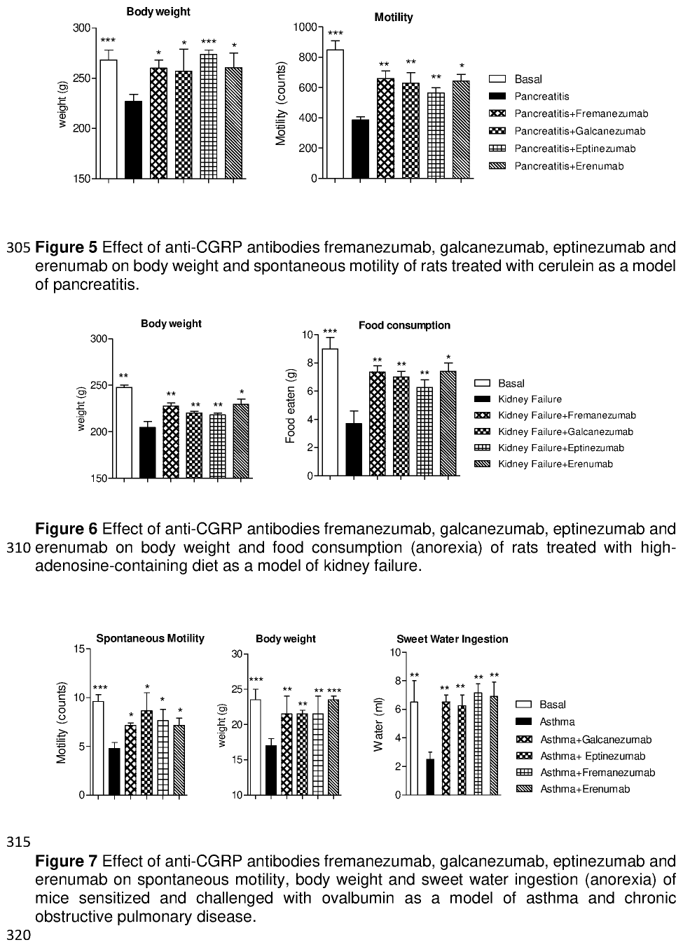

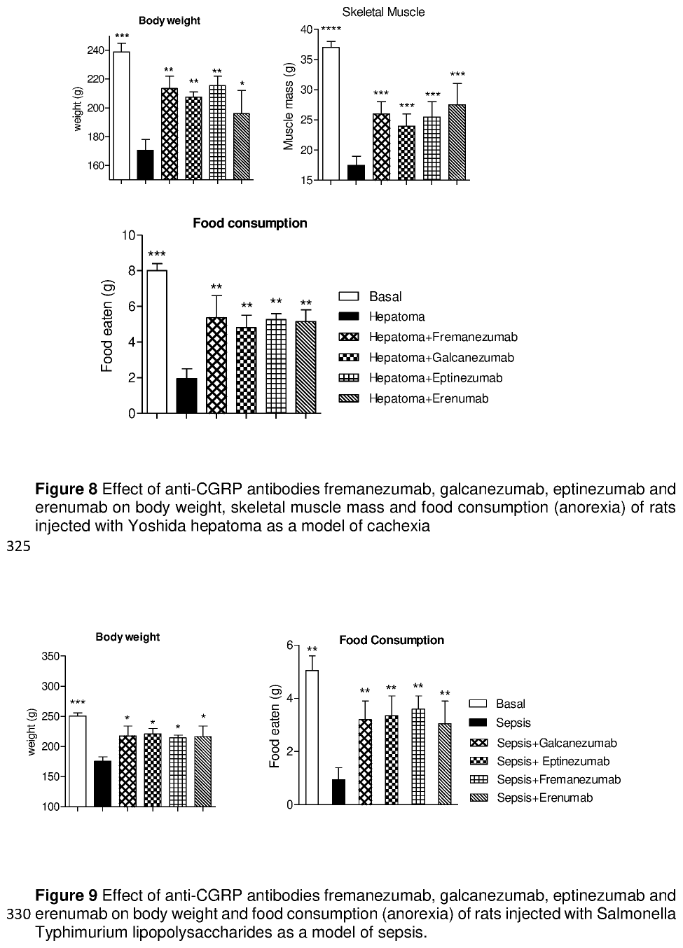

### BEST MODE FOR CARRYING OUT THE INVENTION

The best mode for carrying out the invention is to treat patients during interoceptive disorders or at risk of interoceptive disorders with weekly or monthly doses of fremanezumab, galcanezumab, eptinezumab or erenumab administered by different routes such as, for example, but not limited to, subcutaneous or intravenous.

1. Anti-CGRP antibodies, anti-CGRP receptor antibodies or their antigen-binding fragments in pharmaceutically acceptable formulations thereof for the use in the prevention and therapy of malaise (negative perception of the health status in an individual).

2. Anti-CGRP antibodies, anti-CGRP receptor antibodies or their antigen-binding fragments in pharmaceutically acceptable formulations thereof for the use in the prevention and therapy of eating disorders or body weight loss.

3. Anti-CGRP antibodies, anti-CGRP receptor antibodies or their antigen-binding fragments in pharmaceutically acceptable formulations thereof for the use to stimulate appetite and body weight gain.

4. Anti-CGRP antibody for use according to claims 1-3 in case said antibody is Fremanezumab.

5. Anti-CGRP antibody for use according to claims 1-3 in case said antibody is Galcanezumab.

6. Anti-CGRP antibody for use according to claims 1-3 in case said antibody is Eptinezumab.

7. Anti-CGRP receptor antibody for use according to claims 1-3 in case said antibody is Erenumab.

8. Anti-CGRP antibodies, anti-CGRP receptor antibodies or their antigen-binding fragments for use according to claims 1-7 in an individual affected by neoplastic (cachexia), inflammatory, autoimmune, rheumatic, respiratory chronic obstructive and fibrotic, gastroenteric, hepatic, pancreatic, abdominal, renal, cardiologic, neurological and psychiatric disorders.

9. Anti-CGRP antibodies, anti-CGRP receptor antibodies or their antigen-binding fragments for use according to claims 1-7 in an individual affected by chronic or postsurgical pain.

10. Anti-CGRP antibodies, anti-CGRP receptor antibodies or their antigen-binding fragments for use according to claims 1-7 in an individual exposed or to be exposed to anti-tumoral therapy (chemotherapy).
